SEQUENCE LISTING (7 sequences)

SEQ ID NO: 1 - DNA, 1860 bp, Homo sapiens
tccgcctgcgctcctcagcctgacggtccgcctttcggggctcctcagccttgtcacccgctcttggttttccttttctcttcatctttggctcctttgaccactcgaagccgcgcagcgggttccagcggacctcacagcagccccagaagtggtgcgccaagcacagcctctgctcctcctcgagccggtcgggaactgctgcctgccgccatcatgtctactgcaaatcctgaaactccaaactcaaccatctccagagaggccagcacccagtcctcatcagctgcaaccagccaaggctatattttaccagaaggcaaaatcatgccaaacactgtttttgttggaggaattgatgttaggatggatgaaactgagattagaagcttctttgctagatatggttcagtgaaagaagtgaagataatcactgatcgaactggtgtgtccaaaggctatggatttgtttcattttttaatgacgtggatgtgcagaagatagtagaatcacagataaatttccatggtaaaaagctgaagctgggccctgcaatcaggaaacaaaatttatgtgcttatcatgtgcagccacgtcctttggtttttaatcatcctcctccaccacagtttcagaatgtctggactaatccaaacactgaaacttatatgcagcccacaaccacgatgaatcctataactcagtatgttcaggcatatcctacttacccaaattcaccagttcaggtcatcactggatatcagttgcctgtatataattatcagatgccaccacagtggcctgttggggagcaaaggagctatgttgtacctccggcttattcagctgttaactaccactgtaatgaagttgatccaggagctgaagttgtgccaaatgaatgttcagttcatgaagctactccaccctctggaaatggcccacaaaagaaatctgtggaccgaagcatacaaacggtggtatcttgtctgtttaatccagagaacagactgagaaactctgttgttactcaagatgactacttcaaggataaaagagtgcatcactttagaagaagtcgggcaatgcttaaatctgtttgatcctcctggcttatctagttacatgggaagttgctggttttgaatattaagctaaaaggtttccactattatagaaattctgaattttggtaaatcacactcaaactttgtgtataagttgtattattagactctctagttttatcttaaactgttcttcattagatgtttatttagaaactggttctgtgttgaaatatagttgaaagtaaaaaaataattgagactgaaagaaactaagatttatctgcaaggattttttaaaaattggcattttaagtgtttaaaagcaaatactgattttcaaaaaaatgtttttaaaaacctattttgaaaggtcagaattttgttggtctgaatacaaacatttcacttctccaacaagtacctgtgaacagtacagtatttacagtattgagctttgcatttatgatttctccagaaatttaccacaaaagcaaaatttttaaaactgcatttttaatcagtggaactcaatatatagttagctttattgaagtcttcttatctaaacccagcaaaacagattcaaagcgaacagtccaatcagtgggtcatatgtttattcaaaatattttatcttttagctagaatccacacatatatatcctatttgattarggtagtaattagataactaaaattctgggcctaattttttaaagaatccmagacaaactaaactttactaggtacataagcttctccatgaatcaccatcctcctttttggtaa

SEQ ID NO: 2 - protein, 295 aa, Homo sapiens
MSTANPETPNSTISREASTQSSSAATSQGYILPEGKIMPNTVFVGGIDVRMDETEIRSFFARYGSVKEVKIITDRTGVSKGYGFVSFFNDVDVQKIVESQINFHGKKLKLGPAIRKQNLCAYHVQPRPLVFNHPPPPQFQNVWTNPNTETYMQPTTTMNPITQYVQAYPTYPNSPVQVITGYQLPVYNYQMPPQWPVGEQRSYVVPPAYSAVNYHCNEVDPGAEVVPNECSVHEATPPSGNGPQKKSVDRSIQTVVSCLFNPENRLRNSVVTQDDYFKDKRVHHFRRSRAMLKSV

SEQ ID NO: 3 - protein, 150 aa, Homo sapiens
PNTETYMQPTTTMNPITQYVQAYPTYPNSPVQVITGYQLPVYNYQMPPQWPVGEQRSYVVPPAYSAVNYHCNEVDPGAEVVPNECSVHEATPPSGNGPQKKSVDRSIQTVVSCLFNPENRLRNSVVTQDDYFKDKRVHHFRRSRAMLKSV

SEQ ID NO: 4 - DNA, 19 bp, Homo sapiens
agccacgtcctttggtttt

SEQ ID NO: 5 - DNA, 19 bp, Homo sapiens
gtgggccatttccagaggg

SEQ ID NO: 6 - DNA, 24 bp, Homo sapiens
cgacgaggcccagagcaagagagg

SEQ ID NO: 7 - DNA, 30 bp, Homo sapiens
cgtcaggcagctcatagctcttctccaggg

(19) United States
(12) Patent Application Publication
(10) Pub. No.: US 2005/0287547 A1
(43) Pub. Date: Dec. 29, 2005

(54) Embryonic stem cell markers and uses thereof

(76) Inventor: Judith Seligman, Raanana (IL)

Correspondence Address: WINSTON & STRAWN LLP, 1700 K STREET, N.W., WASHINGTON, DC, 20006, US

(21) Appl. No.: 11/041,070

(22) Filed: Jan. 21, 2005

Related U.S. Application Data
(63) Continuation of application No. PCT/IL03/00605, filed on Jul. 23, 2003.
(60) Provisional application No. 60/397,601, filed on Jul. 23, 2002.

(51) Int. Cl. C12Q 1/68; C12N 5/08

(52) U.S. Cl. 435/6; 435/366

(57) ABSTRACT

The present invention provides methods and reagents for identification, separation and characterization of embryonic stem cells using markers previously recognized as germ cell specific. One currently preferred marker is the DAZL marker now shown to be expressed in multipotent and pluripotent stem cells. Antibodies and other agents, capable of binding the novel marker and useful for selecting these stem cells are also provided as well as diagnostic and therapeutic applications of pluripotent stem cells selected according to the present invention.

### CROSS-REFERENCE TO RELATED APPLICATIONS

This application is a continuation of International application PCT/IL2003/000605 filed 23 Jul. 2003 and claims the benefit of U.S. provisional application 60/397,601 filed 23 Jul. 2002. The entire content of each application is expressly incorporated herein by reference thereto.

### FIELD OF THE INVENTION

The present invention relates to methods of identification, characterization and separation of pluripotent or multipotent stem cells from tissues, body fluids, cell cultures and cell suspensions by means of markers expressed selectively in stem cells. Specifically, the present invention discloses that markers, recognized previously as germ stem cell markers, are useful also as embryonic stem cell markers, as exemplified herein using the DAZL marker. The present invention further relates to diagnostic and therapeutic uses of pluripotent or multipotent stem cells identified, characterized or separated using these stem cell markers.

### BACKGROUND OF THE INVENTION

Stem cells are undifferentiated cells which can give rise to a succession of mature functional cells. For example, a hematopoietic stem cell may give rise to any of the different types of terminally differentiated blood cells. Embryonic stem (ES) cells are derived from the embryo and are pluripotent, thus possessing the capability of developing into any organ or tissue type or, at least potentially, into a complete embryo.

The first evidence for the existence of stem cells came from studies of embryonic carcinoma (EC) cells, the undifferentiated stem cells of teratocarcinomas, which are tumors derived from germ cells. These cells were found to be pluripotent and immortal, but possess limited developmental potential and abnormal karyotypes (Rossant and Papaioannou, Cell Differ 15,155-161, 1984). ES cells, on the other hand, are thought to retain greater developmental potential because they are derived from normal embryonic cells, without the selective pressures of the teratocarcinoma environment.

Pluripotent embryonic stem cells have traditionally been derived principally from two embryonic sources. One type can be isolated in culture from cells of the inner cell mass of a pre-implantation embryo and are termed embryonic stem (ES) cells (Evans and Kaufman, Nature 292,154-156, 1981; U.S. Pat. No. 6,200,806). A second type of pluripotent stem cell can be isolated from primordial germ cells (PGCS) in the mesenteric or genital ridges of embryos and has been termed embryonic germ cell (EG) (U.S. Pat. No. 5,453,357, U.S. Pat. No. 6,245,566). Both human ES and EG cells are pluripotent. This has been shown by differentiating cells in vitro and by injecting human cells into immunocompromised (SCUM) mice and analyzing resulting teratomas (U.S. Pat. No. 6,200,806).

Human ES, EG and EC cells, as well as primate ES cells, express alkaline phosphatase, the stage-specific embryonic antigens SSEA-3 and SSEA-4, and surface proteoglycans that are recognized by the TRA-1-60; and TRA-1-81 antibodies. All these markers typically stain these cells, but are not entirely specific to stem cells, and thus cannot be used to isolate stem cells from organs or peripheral blood.

Methods for separation and use of hematopoetic stem cells are known in the art.

Characterizations and isolation of hematopoietic stem cells are reported in U.S. Pat. No. 5,061,620. The hematopoietic CD34 marker is the most common marker known to identify specifically blood stem cells, and CD34 antibodies are used to isolate stem cells from blood for transplantation purposes. However, CD34+ cells can differentiate only to blood cells and differ from embryonic stem cells which have the capability of developing into different body cells. Moreover, expansion of CD34+ cells is limited as compared to embryonic stem cells which are immortal. U.S. Pat. No. 5,677,136 discloses a method for obtaining human hematopoietic stem cells by enrichment for stem cells using an antibody which is specific for the CD59 stem cell marker. The CD59 epitope is highly accessible on stem cells and less accessible or absent on mature cells. U.S. Pat. No. 6,127,135 provides an antibody specific for a unique cell marker (EM10) that is expressed on stem cells, and methods of determining hematopoietic stem cell content in a sample of hematopoietic cells. These disclosures are specific for hematopoietic cells and the markers used for selection are not absolutely absent on more mature cells.

There have been great efforts toward isolating pluripotent or multipotent stem cells, in earlier differentiation stages than hematopoietic stem cells, in substantially pure or pure form for diagnosis, replacement treatment and gene therapy purposes. Stem cells are important targets for gene therapy, where the inserted genes are intended to promote the health of the individual into whom the stem cells are transplanted. In addition, the ability to isolate stem cells may serve in the treatment of lymphomas and leukemias, as well as other neoplastic conditions where the stem cells are purified from tumor cells in the bone marrow or peripheral blood, and reinfused into a patient after myelosuppressive or myeloablative chemotherapy.

Multiple adult stem cell populations have been discovered from various adult tissues. In addition to hematopoietic stem cells, neural stem cells were identified in adult mammalian central nervous system (Ourednik et al. Clin. Genet. 56, 267, 1999). Adult stem cells have also been identified from epithelial and adipose tissues (Zuk et al. Tissue Engineering 7, 211, 2001). Mesenchymal stem cells (MSCs) have been cultured from many sources, including liver and pancreas (Hu et al. J. Lab Clin Med. 141, 342-349, 2003). Recent studies have demonstrated that certain somatic stem cells appear to have the ability to differentiate into cells of a completely different lineage (Pfendler KC and Kawase E, Obstet Gynecol Surv 58, 197-208, 2003). Monocyte derived (Zhao et al. Proc. Natl. Acad. Sci. USA 100, 2426-2431, 2003) and mesodermal derived (Schwartz et al. J. Clin. Invest 109, 1291-1301, 2002) cells that possess some multipotent characteristics were identified. The presence of multipotent “embryonic-like” progenitor cells in blood was suggested also by in-vivo experiments following bone marrow transplantations (Zhao et al. Brain Res Protoc 11, 38-45, 2003). However, such multipotent “embryonic-like” stem cells cannot be identified and isolated using the known markers.

The possibility of recovering fetal cells from the maternal circulation has generated interest as a possible means, non-invasive to the fetus, of diagnosing fetal anomalies (Simpson and Elias, J. Am. Med. Assoc. 270, 2357-2361, 1993). Prenatal diagnosis is carried out widely in hospitals throughout the world. Existing procedures such as fetal, hepatic or chorionic biopsy for diagnosis of chromosomal disorders including Down's syndrome, as well as single gene defects including cystic fibrosis are very invasive and carry a considerable risk to the fetus. Amniocentesis, for example, involves a needle being inserted into the womb to collect cells from the embryonic tissue or amniotic fluid. The test, which can detect Down's syndrome and other chromosomal abnormalities, carries a miscarriage risk estimated at 1%. Fetal therapy is in its very early stages and the possibility of early tests for a wide range of disorders would undoubtedly greatly increase the pace of research in this area. Thus, relatively non-invasive methods of prenatal diagnosis are an attractive alternative to the very invasive existing procedures. A method based on maternal blood should make earlier and easier diagnosis more widely available in the first trimester, increasing options to parents and obstetricians and allowing for the eventual development of specific fetal therapy.

Initial interest was directed towards trophoblastic detection systems but separation of those cells by flow cytometry has been unreliable, as the maternal lymphocytes appear to absorb proteins released by trophoblastic cells (Mueller et al. Lancet 336, 197-200, 1990). More recently, attention has focused on the development of methods to isolate fetal blood cells for cytogenetic analysis, particularly nucleated fetal erythrocytes as their numbers exceed those of fetal lymphocytes in the maternal circulation. Identification of fetal red blood cells in maternal blood has been described during pregnancy with a male fetus using Y-specific DNA sequences (Cheung et al. Nature Genetics 14, 264-268, 1996; and Williamson, Nature Genetics 14, 239-249, 1996) and by karyotype identification in trisomic conditions (for example, Bianchi et al. Hum. Genet. 90, 368-370, 1992).

Various methods for identifying fetal red blood cells in maternal blood have been proposed with no success for carrying out a reliable prenatal diagnosis. For example, identification of fetal nucleated red blood cells using combined immunocytochemistry, human fetal haemoglobin antibody and an in situ hybridisation method using X and Y chromosome probes has been suggested (Pazouki et al. Acta Histochem. (Jena) 98, 29-37, 1996). Wachtel et al. (Human Reproduction 6, 1466-1469, 1991) described the use of PCR to identify Y-specific DNA sequences in maternal cells isolated by cell sorting with transferrin receptor antibody and glycophorin A antibody. Yeoh et al. (Prenatal Diagnosis 11, 117-123, 1991) described the detection of fetal cells in the maternal circulation by enzymatic amplification of a single copy gene that was fetal specific. Holzgreve et al. (J. Reprod. Med. 37, 410-418, 1992) showed that the transferrin receptor antigen alone is not sufficient for enrichment of fetal nucleated erythrocytes and points out that the reproducibility and reliability of the techniques are still limited, mainly due to the lack of very specific cell markers. The use of anti-CD71 is reviewed by Williamson (Nature Genetics 14, 239-240, 1996). The problems with this approach include having to look at thousands of cells to find a few fetal ones and the fact that anti-CD71 antibodies are not selective enough for fetal cells and do not react with embryonic cells. Zheng et al (J. Med. Genet. 30, 1051-1056, 1993) described the use of a magnetic activated cell sorter (MACS) to enrich fetal nucleated erythrocytes using mouse monoclonal antibodies specific for CD45 and CD32 to deplete leucocytes from maternal blood. The article pointed out that significant maternal contamination was present even after MACS enrichment.

The DAZL gene, known also as DAZL1, DAZLA or DAZH, is an autosomal homolog of the DAZ (Deletion in Azoospermia) gene present on the Y chromosome (Saxena, R. et al. Nature Genet. 14, 292-299, 1996). These genes encode RNA binding proteins, found to be expressed specifically in germ cells in the testis. Later studies have demonstrated that the DAZL gene expression is unique as it is expressed before meiosis in male and female gonads (Seligman and Page, Biochem. Biophys. Res. Corn. 245, 878-82, 1998). This pattern of expression suggests that these genes participate in the early proliferation, differentiation and maintenance of male and female germ cells. Expression studies of a DAZL homolog in the mouse, denoted Dazl, suggest that this gene is expressed as early as when primordial germ cells appear in the developing embryonic gonads. The similarity between the Dazl and DAZL expression in male and female gonads suggests that DAZL gene is expressed in early human gonad development as well, presumably in primordial germ cells.

Numerous genes are known to be expressed exclusively in male or female germ cells, mainly in meiotic or postmeiotic cells, but not in the earliest stages of gametogenesis. The expression of the human DAZL gene in both male and female germ cells so early during embryonic development is unusual. In the mouse, only a very few genes are known to be expressed exclusively in male and female germ cells early during gametogenesis, but no human homologous genes were studied. The mouse germ cell nuclear antigen (GCNA1) is expressed in primordial germ cells, and later in oogonia and prospermatogonia, as is the DAZL gene, but no DNA sequences of this antigen are available (Endres and May, Dev. Biol. 163, 331-340, 1994). The TIAR gene, which is also an RNA-binding protein such as Dazl, was found to be expressed in primordial germ cells (Beck, A. R. P. et al. Proc. Natl. Acad. Sci. USA 95, 2331-2336, 1998).

U.S. Pat. Nos. 5,695,935; 5,871,920 and 6,020,476 disclose the nucleotide sequences of the DAZ gene family associated with azospermia, while WO 02/10203 and US Application publication no. 2002165142 disclose four additional DAZ genes on the Y chromosome, isolated polypeptides encoded by these genes, antibodies to these polypeptides and methods for analyzing samples for the presence of the disclosed genes and their protein products.

Recently (Moore et al. Proc. Natl. Acad. Sci. USA 100, 538-543, 2003), PUM2, a human homolog of Pumilio (a protein required to maintain germ line stem cells in Drosophila and Caenorhabditis elegans), has been identified as a protein that forms a stable complex with DAZ through the same functional domain required for RNA binding and protein-protein interactions. It was shown that PUM2 is expressed predominantly in human embryonic stem cells and germ cells and co-localizes with DAZ and DAZL in germ cells, suggesting that PUM2 is a component of conserved cellular machinery that may be required for germ cell development.

Nowhere in the prior art is it disclosed or suggested that the DAZL protein is also expressed in embryonic stem cells, the progenitors of the primordial germ cells, and that it can be used as a unique marker for identification, separation and characterization of stem cells in adult organs, tissue culture and cell suspensions. There exists an unmet need for improved methods for identifying stem cells among adult cell populations. In addition there exists an unmet need for improved methods for identifying fetal cells in maternal blood. In order to carry out mutation analysis and diagnose genetic diseases, a very pure fetal cell fraction is needed. It would be very advantageous to have a specific fetal or stem cell marker, which does not react with other maternal cells.

### SUMMARY OF THE INVENTION

The present invention provides methods of identifying, characterizing and separating stem cells having characteristics of embryonic stem (ES) cells for diagnostic, therapy and tissue engineering. In particular, the present invention provides methods of identifying, selecting and separating embryonic stem cells or fetal cells from maternal blood and to reagents for use in prenatal diagnosis and tissue engineering methods. The present invention provides for the first time a specific marker that can be used for identification, separation and characterization of valuable stem cells from tissues and organs, overcoming the ethical and logistical difficulties in the currently available methods for obtaining embryonic stem cells.

The present invention overcomes the limitations of known markers for identification and separation of embryonic or fetal stem cells by disclosing a very specific type of marker, which does not react with differentiated somatic maternal cell types. The present invention discloses for the first time that markers previously identified as primordial germ cell or germ stem cell markers are in fact expressed in embryonic stem cells and other stem cell types.

By way of exemplification, the marker denoted DAZL is now disclosed as useful for identifying, selecting and isolating pluripotent or multipotent stem cells including embryonic stem cells, which have the capability of differentiating into varied cell lineages.

According to one aspect of the present invention a novel method for identifying pluripotent or multipotent stem cells in peripheral blood and other organs is disclosed. According to this aspect an embryonic stem cell marker is selected based on its selective expression in primordial germ cells and/or germ stem cells and its absence in differentiated somatic cells. Thus, genes and proteins expressed in germ stem cells and in their progenitors, embryonic stem cells, are used according to the present invention as selective markers for isolation of pluripotent or multipotent stem cells from blood, tissue and organs. Preferably the blood cells and tissue samples are of mammalian origin, more preferably human origin.

According to a specific embodiment the present invention provides a method for identifying a selective embryonic stem cell marker comprising the steps of:
i. selecting a germ stem cell marker exhibiting specific expression in germ cells and absence of expression in differentiated somatic cells; and
ii. confirming the expression of said stem cell marker in ES cells.

According to one embodiment of the present invention an embryonic stem cell marker is provided for identifying fetal cells in maternal blood. According to another embodiment of the present invention methods of using the embryonic stem cell marker for identifying, selecting or isolating pluripotent stem or multipotent cells, and analyzing them for fetal abnormalities or for gene therapy and tissue engineering purposes, are disclosed.

According to a currently preferred embodiment of the present invention DAZL is disclosed as a marker of embryonic stem cells. The method of identifying, selecting or isolating stem cells relies on the use of a binding agent to the DAZL transcript or protein.

Use of any agent that is capable of binding to or associating with the DAZL gene or DAZL transcripts or DAZL protein in stem cells for use as a marker for separation of these cells from body fluids, tissues and cell suspensions is within the scope of the present invention. Preferred agents capable of binding the DAZL gene products include proteins that bind to the DAZL protein (e.g., PUM-2 and dazap1) and antibodies that bind to the DAZL protein, as well as oligonucleotide and polynucleotide probes that bind to the gene transcripts (messenger RNA or mRNA). Other binding agents include aptamers capable of binding RNA or proteins.

According to a further aspect of the present invention a polynucleotide or an oligonucleotide sequence which hybridizes to the polynucleotide sequence having SEQ ID NO:1, or fragments of said polynucleotide sequence, are disclosed. The invention further provides a polynucleotide or an oligonucleotide sequence comprising the complement of the polynucleotide sequence of SEQ ID NO:1, or fragments or variants of said polynucleotide sequence.

The binding agents of the present invention also include a polynucleotide or an oligonucleotide that hybridizes to the mRNA sequence transcribed from SEQ ID NO:1.

According to one embodiment the present invention comprises use of a DAZL polynucleotide probe having SEQ ID NO:1 or a polynucleotide having at least 70% homology, preferably at least 80%, more preferably at least 90% homology to said sequence or a fragment thereof.

According to still another embodiment of the present invention there is provided an oligonucleotide of at least 10 bases specifically hybridizable with the DAZL mRNA.

Another embodiment according to the present invention provides methods for selection of pluripotent or multipotent stem cells using the DAZL RNA as a marker. Labeled oligonucleotide or polynucleotide probes or antibodies, or other agents which selectivity bind said RNA may be used for labeling DAZL positive cells and separating them using methods known in the art.

Use of any agent that is capable of binding to the marker DAZL in stem cells for separation of these cells from body fluids and tissues is within the scope of the present invention. Agents which are capable of binding DAZL protein are preferably selected from the group consisting of: a monoclonal antibody, a polyclonal antibody, an active antibody fragment, a protein, a peptide, a peptide analog, a nucleic acid chain, a small organic molecule and an aptamer. The design, identification or selection of a DAZL-binding moiety may be facilitated using computerized molecular modeling methods.

According to a certain embodiment of the present invention methods of using antibodies specific to the DAZL protein, for detection and separation of stem cell are disclosed. These antibodies are capable of recognizing at least one epitope of the DAZL protein and are useful in all separation methods known in the art, specifically for separating stem cells expressing DAZL from other types of cells.

The DAZL specific antibodies, preferably monoclonal antibodies, can be used to separate specific stem cells by separation methods known in the art. One embodiment according to the present invention discloses the use of immunofluorescent techniques.

According to one aspect of the present invention there is provided an antibody comprising at least the antigen binding portion of an immunoglobulin specifically recognizing and binding a polypeptide having at least 70% homology, preferably at least 80%, more preferably at least 90% or more homology to SEQ ID) NO: 2 or any peptide fragment thereof retaining antigenic specificity of at least one epitope of DAZL. According to a specific embodiment of this aspect of the present invention the antibody specifically recognizes and binds to a polypeptide comprising contiguous amino acids having at least 70%, preferably at least 80%, more preferably at least 90% homology to SEQ ID NO:3 or any peptide fragment thereof retaining the antigenic specificity of at least one epitope of DAZL.

The present invention further provides adult stem cells obtained from body tissues, including but not limited to blood and bone marrow, using their expression of DAZL for isolation. Selected pluripotent or multipotent stem cells according to the invention may be used for any diagnostic, prophylactic, therapeutic or research purpose known in the art.

By way of a non-limiting example, hematopoietic stem cells selected using the marker DAZL may be used in regenerating the hematopoietic system of a host deficient in any class of hematopoietic cells. A host that is diseased can be treated by removal of bone marrow, isolation of stem cells and treatment with drugs or irradiation prior to re-engraftment of stem cells. The novel markers of the present invention may be used for identifying and isolating various hematopoietic cells; detecting and evaluating growth factors relevant to stem cell self-regeneration; the development of hematopoietic cell lineages; and assaying for factors associated with hematopoietic development.

Another embodiment according to the present innovation provides methods of isolation of adult pluripotent or multipotent stem cells from peripheral blood following granulocyte-colony stimulating factor (G-CSF) treatment for preservation proposes. These stem cells can be preserved by methods known in the art and maintained in an adult stem cell bank for future use.

According to one embodiment, the separated stem cells are used in methods of gene therapy. Cells are modified by appropriate gene transfer, to correct genetic defects or provide genetic capabilities naturally lacking in the stem cells or their progeny.

According to another embodiment the separated stem cells are used to study stem cell differentiation. Stem cells having the capacity to differentiate to various cell types are useful in tissue engineering and regeneration techniques.

Another aspect of the present invention is directed to kits for enrichment and detection of fetal cells within a blood specimen, e.g., maternal or umbilical cord blood. Such a kit comprises at least one reagent selected from an antibody or antibody fragment specific for DAZL protein and a probe specific for DAZL RNA, and instructions for stem cell detection or instructions for performing stem cell enrichment. Such a kit may further comprise means for separating fetal cells as exemplified by one or more reagents to prepare a density gradient that concentrates fetal cells.

Essentially all of the uses known or envisioned in the prior art for stem cells, can be accomplished with the molecules of the present invention. These uses include diagnostic, prophylactic and therapeutic techniques.

### BRIEF DESCRIPTION OF THE DRAWINGS

FIG. 1. DAZL expression in embryonic stem cells determined by RT-PCR. RNAs were extracted from ES cells, MEF mouse fibroblast feeder layer, FHs173 human fibroblast cell line and human embryonic kidney and spleen. Specific primers were used to amplify the DAZL transcript. The gene encoding beta-actin was used as a reference reaction.

FIG. 2. Immunofluorescence of DAZL expression in bone marrow cells. Fixed bone marrow cells were incubated with preimmune serum (not shown) or DAZL antibodies. Left, DAZL expression, fluorescent illumination; Right, light illumination.

FIG. 3. DAZL expression in G-CSF mobilized mononuclear blood cells by RT-PCR. Lane 1, G-CSF mobilized mononuclear cells; Lane 2, FHsl73 fibroblast. DAZL, amplification of DAZL transcript; Actin, amplification of β-actin gene.

FIG. 4. FACS analysis of DAZL labeled G-CSF mononuclear cells. Cells incubated with DAZL antibodies (left) and preimmune serum (right) were analyzed using a fluorescence activated cell sorter (FACS). The subset of cells expressing DAZL is selected in a box. The lower figure represents a graph of the analyzed cell population.

FIG. 5. DAZL expression in G-CSF mobilized mononuclear cells. Slides were incubated with preimmune serum or DAZL antibodies, washed and stained with FITC-conjugated anti-rabbit IgG. Slides were analyzed by using an Olympus fluorescent microscope. Left, fluorescent illumination; Right, light illumination.

### DETAILED DESCRIPTION OF THE INVENTION

Terminology and Definitions

Stem cells are undifferentiated cells, which can give rise to a succession of mature functional cells. Embryonic stem (ES) cells are pluripotent, thus possessing the capability of developing into any organ or tissue type or, at least potentially, into a complete embryo. Adult stem cells are stem cells derived from tissues, organs or blood of an adult organism. The term embryonic-like stem cells refer to cells derived from tissues, organs or blood, possessing pluripotent characteristics of embryonic stem cells.

As used herein, the term “pluripotent stem cells” refers to cell that are: (i) capable of indefinite proliferation in vitro in an undifferentiated state; (ii) maintain a normal karyotype through prolonged culture; and (iii) maintain the potential to differentiate to derivatives of all three embryonic germ layers (endoderm, mesoderm, and ectoderm) even after prolonged culture.

The term “multipotent cells” known also as “multipotent adult progenitor cells (MAPCs)” refers to adult stem cells which can give rise to a limited number of particular types of cells. For example, hematopoietic stem cells in the bone marrow are multipotent and give rise to the various types of blood cells.

The term “primordial germ cells” (PGCs) is used to describe undifferentiated embryonic germ cells isolated over a period of time post-fertilization from anlagen or from yolk sac, mesenteries, or gonadal ridges of embryos/fetus. Gonocytes of later testicular stages also can be useful sources of PGCs. PGCs are the source from which embryonic germ cells are derived and are pluripotent.

The term “germ cell specific gene expression” and “germ cell selective gene expression” are used interchangeably and refer to genes that are expressed in testis or ovary germ cells and are absent in other organs and in differentiated somatic cells types.

The term “undetectable expression” or “absence of expression” are used interchangeably, and with respect to detection of stem cell marker refers to negative expression results obtained by methods known in the art including but not limited to Northern-blotting, RT-PCR, Western blotting and immunohistochemistry.

The term “homology”, as used herein, refers to a degree of sequence similarity in terms of shared amino acid or nucleotide sequences. There may be partial homology or complete homology (i.e., identity).

The terms “complementary” or “complementarity”, as used herein, refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between the single stranded molecules. The degree of complementarity between nucleic acid strands has significant effects on the efficiency and strength of hybridization between nucleic acid strands.

As used herein in the specification and in the claims section that follows, the phrase “complementary polynucleotide sequence” or complementary DNA (cDNA) includes sequences which result from reverse transcription of a messenger RNA template using a reverse transcriptase or any other RNA dependent DNA polymerase. Such sequences can be subsequently amplified in vivo or in vitro using a DNA dependent DNA polymerase.

A partially complementary sequence that at least partially inhibits an identical sequence from hybridizing to a target nucleic acid is referred to using the functional term “substantially homologous.” A substantially homologous sequence or hybridization probe will compete for and inhibit the binding of a completely homologous sequence to the target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity). In the absence of non-specific binding, the probe will not hybridize to the second non-complementary target sequence.

The terms “stringent conditions” or “stringency”, as used herein, refer to the conditions for hybridization as defined by the nucleic acid, salt, and temperature. These conditions are well known in the art and may be altered in order to identify or detect identical or related polynucleotide sequences. Numerous equivalent conditions comprising either low or high stringency depend on factors such as the length and nature of the sequence (DNA, RNA, base composition), nature of the target (DNA, RNA, base composition), milieu (in solution or immobilized on a solid substrate), concentration of salts and other components (e.g., formamide, dextran sulfate and/or polyethylene glycol), and temperature of the reactions (within a range from about 5° C. below the melting temperature (Tm) of the probe to about 20° C. to 25° C. below the melting temperature). One or more factors may be varied to generate conditions of either low or high stringency different from, but equivalent to, the above listed conditions.

“Nucleic acid sequence” as used herein refers to an oligonucleotide, nucleotide, or polynucleotide, and fragments thereof, and to DNA or RNA of genomic or synthetic origin which may be single- or double-stranded, and represent the sense or antisense strand. “Fragments” are those nucleic acid sequences which are greater than 60 nucleotides than in length, and most preferably includes fragments that are at least 100 nucleotides in length.

The term “oligonucleotide” refers to a nucleic acid sequence of at least about 6 nucleotides to about 60 nucleotides, preferably about 10 to 50 nucleotides, and more preferably about 20 to 30 nucleotides, which can be used in PCR amplification or a hybridization assay, or a microarray. As used herein, oligonucleotide is substantially equivalent to the terms “amplimers”, “primers”, “oligomers”, and “probes”, as commonly defined in the art.

The terms “specific binding” or “specifically binding”, as used herein, refers to that interaction for example between a protein or peptide and a binding agent such as an antibody. The interaction is dependent upon the presence of a particular structure (i.e., the antigenic determinant or epitope) of the protein recognized by the binding molecule. For example, if an antibody is specific for epitope “A”, the presence of a protein containing epitope A (or free, unlabeled A) will reduce the amount of labeled A bound to the antibody.

The term “antigenic determinant”, as used herein, refers to that fragment of a molecule (i.e., an epitope) that makes contact with a particular antibody. When a protein or fragment of a protein is used to immunize a host animal, numerous regions of the protein may induce the production of antibodies which bind specifically to a given region or three-dimensional structure on the protein; these regions or structures are referred to as antigenic determinants. An antigenic determinant may compete with the intact antigen (i.e., the immunogen used to elicit the immune response) for binding to an antibody.

As used herein, the terms “antibody” or “antibodies” include the entire antibody and antibody fragments containing functional portions thereof. The term “antibody” includes any monospecific or bispecific compound comprised of a sufficient portion of the light chain variable region and/or the heavy chain variable region to effect binding to the epitope to which the whole antibody has binding specificity. The fragments can include the variable region of at least one heavy or light chain immunoglobulin polypeptide, and include, but are not limited to, Fab fragments, F(ab′)₂ fragments, Fv fragments and scFv.

In addition, the monospecific domains can be attached by any method known in the art to another suitable molecule. The attachment can be, for instance, chemical or by genetic engineering. Antibodies according to the present invention can be produced by any recombinant means known in the art. Such recombinant antibodies include, but are not limited to, fragments produced in bacteria and non-human antibodies in which the majority of the constant regions have been replaced by human antibody constant regions. In addition, such “humanized” antibodies can be obtained by host vertebrates genetically engineered to express the recombinant antibody.

The antibodies according to the present invention are obtained by methods known in the art for production of antibodies or functional portions thereof. Such methods include, but are not limited to, separating B cells with cell-surface antibodies of the desired specificity, cloning the DNA expressing the variable regions of the light and heavy chains and expressing the recombinant genes in a suitable host cell. Standard monoclonal antibody generation techniques can be used wherein the antibodies are obtained from immortalized antibody-producing hybridoma cells. These hybridomas can be produced by immunizing animals with stem cells, and fusing B lymphocytes from the immunized animals, preferably isolated from the immunized host spleen, with compatible immortalized cells, preferably a B cell myeloma.

Methods for the generation and selection of monoclonal antibodies are well known in the art, as summarized for example in reviews such as Tramontano and Schloeder, (Methods in Enzymology 178, 551-568, 1989). A recombinant or synthetic DAZL or a portion thereof of the present invention may be used to generate antibodies in vitro. More preferably, the recombinant or synthetic DAZL of the present invention is used to elicit antibodies in vivo. In general, a suitable host animal is immunized with the recombinant or synthetic DAZL of the present invention or a portion thereof including at least one continuous or discontinuous epitope. Advantageously, the animal host is a mouse of an inbred strain. Animals are typically immunized with DAZL or portion thereof in a physiologically acceptable vehicle, and a suitable adjuvant, which achieves an enhanced immune response to the immunogen. By way of example, the primary immunization conveniently may be accomplished with DAZL or a portion thereof and Freund's complete adjuvant, said mixture being prepared in the form of a water-in-oil emulsion. Typically the immunization may be administered to the animals intramuscularly, intradermally, subcutaneously, intraperitoneally, into the footpads, or by any appropriate route of administration. The immunization schedule of the immunogen may be adapted as required, but customarily involves several subsequent or secondary immunizations using a milder adjuvant such as Freund's incomplete adjuvant. Antibody titers and specificity of binding can be determined during the immunization schedule by any convenient method including by way of example radioimmunoassay, or enzyme linked immunosorbant assay, which is known as the ELISA assay. When suitable antibody titers are achieved, antibody producing lymphocytes from the immunized animals are obtained, and these are cultured, selected and closed, as is known in the art. Typically, lymphocytes may be obtained in large numbers from the spleens of immunized animals, but they may also be retrieved from the circulation, the lymph nodes or other lymphoid organs. Lymphocyte are then fused with any suitable myeloma cell line, to yield hybridomas, as is well known in the art. Alternatively, lymphocytes may also be stimulated to grow in culture; and may be immortalized by methods known in the art including the exposure of these lymphocytes to a virus; a chemical or a nucleic acid such as an oncogene, according to established protocols. After fusion, the hybridomas ate cultured under suitable culture conditions, for example in multiwell plates, and the culture supernatants are screened to identify cultures containing antibodies that recognize the hapten of choice. Hybridomas that secrete antibodies that recognize the recombinant or synthetic DAZL are cloned by limiting dilution and expanded, under appropriate culture conditions. Monoclonal antibodies are purified and characterized in terms of immunoglobulin type and binding affinity.

The antibodies can be conjugated to other compounds including, but not limited to, enzymes, magnetic beads, colloidal magnetic beads, haptens, fluorochromes, metal compounds, radioactive compounds or drugs. The enzymes that can be conjugated to the antibodies include, but are not limited to, alkaline phosphatase, peroxidase, urease and β-galactosidase. The fluorochromes that can be conjugated to the antibodies include, but are not limited to, fluorescein isothiocyanate, tetramethylrhodamine isothiocyanate, phycoerythrin, allophycocyanins and Texas Red. The metal compounds that can be conjugated to the antibodies include, but are not limited to, ferritin, colloidal gold, and particularly, colloidal superparamagnetic beads. The haptens that can be conjugated to the antibodies include, but are not limited to, biotin, digoxigenin, oxazalone, and nitrophenol. The radioactive compounds that can be conjugated or incorporated into the antibodies are known to the art, and include but are not limited to technetium 99m (⁹⁹Tc) ¹²⁵I and amino acids comprising any radionuclides, including, but not limited to, ¹⁴C, ³H and ³⁵S.

The term DAZL refers to “DAZ-like autosomal” or “Deleted in azoospermia-like 1” for designation of a polynucleotide or amino acid sequence is interchangeable with any of the terms DAZL1, DAZLA and DAZH. The human DAZL gene is the polynucleotide sequence of GenBank Accession Number U21663. The cDNA sequence of the human DAZL mRNA (Saxena et al. Nat. Genet. 14, 292-299, 1996), is presented in SEQ ID NO:1, wherein the coding sequence spans nucleotides 217-1098:

| SEQ ID NO:1 | |
|---|---|
| 1 | tccgcctgcg ctcctcagcc tgacggtccg cctttcgggg ctcctcagcc ttgtcacccg |
| 61 | ctcttggttt tccttttctc ttcatctttg gctcctttga ccactcgaag ccgcgcagcg |
| 121 | ggttccagcg gacctcacag cagccccaga agtggtgcgc caagcacagc ctctgctcct |
| 181 | cctcgagccg gtcgggaact gctgcctgcc gccatcatgt ctactgcaaa tcctgaaact |
| 241 | ccaaactcaa ccatctccag agaggccagc acccagtcct catcagctgc aaccagccaa |
| 301 | ggctatattt taccagaagg caaaatcatg ccaaacactg tttttgttgg aggaattgat |
| 361 | gttaggatgg atgaaactga gattagaagc ttctttgcta gatatggttc agtgaaagaa |
| 421 | gtgaagataa tcactgatcg aactggtgtg tccaaaggct atggatttgt ttcatttttt |
| 481 | aatgacgtgg atgtgcagaa gatagtagaa tcacagataa atttccatgg taaaaagctg |
| 541 | aagctgggcc ctgcaatcag gaaacaaaat ttatgtgctt atcatgtgca gccacgtcct |
| 601 | ttggttttta atcatcctcc tccaccacag tttcagaatg tctggactaa tccaaacact |
| 661 | gaaacttata tgcagcccac aaccacgatg aatcctataa ctcagtatgt tcaggcatat |
| 721 | cctacttacc caaattcacc agttcaggtc atcactggat atcagttgcc tgtatataat |
| 781 | tatcagatgc caccacagtg gcctgttggg gagcaaagga gctatgttgt acctccggct |
| 841 | tattcagctg ttaactacca ctgtaatgaa gttgatccag gagctgaagt tgtgccaaat |
| 901 | gaatgttcag ttcatgaagc tactccaccc tctggaaatg gcccacaaaa gaaatctgtg |
| 961 | gaccgaagca tacaaacggt ggtatcttgt ctgtttaatc cagagaacag actgagaaac |
| 1021 | tctgttgtta ctcaagatga ctacttcaag gataaaagag tgcatcactt tagaagaagt |
| 1081 | cgggcaatgc ttaaatctgt ttgatcctcc tggcttatct agttacatgg gaagttgctg |
| 1141 | gttttgaata ttaagctaaa aggtttccac tattatagaa attctgaatt ttggtaaatc |
| 1201 | acactcaaac tttgtgtata agttgtatta ttagactctc tagttttatc ttaaactgtt |
| 1261 | cttcattaga tgtttattta gaaactggtt ctgtgttgaa atatagttga aagtaaaaaa |
| 1321 | ataattgaga ctgaaagaaa ctaagattta tctgcaagga ttttttaaaa attggcattt |
| 1381 | taagtgttta aaagcaaata ctgattttca aaaaaatgtt tttaaaaacc tattttgaaa |
| 1441 | ggtcagaatt ttgttggtct gaatacaaac atttcacttc tccaacaagt acctgtgaac |
| 1501 | agtacagtat ttacagtatt gagctttgca tttatgattt ctccagaaat ttaccacaaa |
| 1561 | agcaaaattt ttaaaactgc atttttaatc agtggaactc aatatatagt tagctttatt |
| 1621 | gaagtcttct tatctaaacc cagcaaaaca gattcaaagc gaacagtcca atcagtgggt |
| 1681 | catatgttta ttcaaaatat tttatctttt agctagaatc cacacatata tatcctattt |
| 1741 | gattarggta gtaattagat aactaaaatt ctgggcctaa ttttttaaag aatccmagac |
| 1801 | aaactaaact ttactaggta cataagcttc tccatgaatc accatcctcc tttttggtaa |

The derived amino acid sequence of the human DAZL protein is presented in SEQ ID NO:2:

| SEQ ID NO:2 | |
|---|---|
| 1 | MSTANPETPN STISREASTQ SSSAATSQGY ILPEGKIMPN TVFVGGIDVR MDETEIRSFF |
| 61 | ARYGSVKEVK IITDRTGVSK GYGFVSFFND VDVQKIVESQ INFHGKKLKL GPAIRKQNLC |
| 121 | AYHVQPRPLV FNHPPPPQFQ NVWTNPNTET YMQPTTTMNP ITQYVQAYPT YPNSPVQVIT |
| 181 | GYQLPVYNYQ MPPQWPVGEQ RSYVVPPAYS AVNYHCNEVD PGAEVVPNEC SVHEATPPSG |
| 241 | NGPQKKSVDR SIQTVVSCLF NPENRLRNSV VTQDDYFKDK RVHHFRRSRA MLKSV |

Certain abbreviations are used herein to describe this invention and the manner of making and using it. For instance, DAZL refers to DAZ-like autosomal and to Deleted in azoospermia-like 1, DNA refers to deoxyribonucleic acid, FACS refers to fluorescence activated cell sorter, FITC refers to fluorescein isothiocyanate, G-CSF refers to granulocytes-colony stimulating factor, PCR refers to polymerase chain reaction, RNA refers to ribonucleic acid, RT-PCR refers to reverse transcriptase PCR.

DAZL Expression in Embryonic Stem Cells

The present invention is directed to the identification and use of markers specific to various lineages of pluripotent or multipotent stem cells, including embryonic stem (ES) cells, fetal stem cells in cord blood or in the maternal circulation and adult stem cells.

It is now disclosed according to the present invention that markers previously taught to be confined to the germ cell lineages, including germ cell precursors, are useful in the identification, characterization, selection or isolation of pluripotent or multipotent stem cells.

As a currently preferred example, DAZL expression has been studied by Reverse transcriptase PCR (RT-PCR) in human embryonic stem cells, teratocarcinoma tumor cells, feeder layer cells and other primary and transformed cell lines. It was found that DAZL is expressed in embryonic stem cells and teratocarcinoma cells, but is not expressed in the differentiated cell lines tested. DAZL thus provides a novel and highly selective marker for stem cell identification and isolation.

Pluripotent or multipotent embryonic-like stem cells may be identified and separated from a plurality of tissues including bone marrow, both adult and fetal, mobilized peripheral blood (MPB), blood, umbilical cord blood, embryonic yolk sac, fetal liver, and spleen, both adult and fetal. Bone marrow cells may be obtained from any known source, including but not limited to, ilium (e.g. from the hip bone via the iliac crest), sternum, tibiae, femora, spine, or other bone cavities.

Adult stem cells, also termed “embryonic-like” stem cells may be also identified, and selected using the DAZL marker according to the present invention. For example, stem cells from epithelial and adipose tissues, neural stem cells from adult mammalian central nervous system, mesenchymal stem cells (MSCs) from liver and pancreas.

ES cells expanding in culture are maintained undifferentiated in order to retain pluripotency. The DAZL marker can be used to monitor ES cell expansion and identify the percentage of non differentiated cells in the cultures.

To assess cell pluripotency, viable cells labeled with a DAZL polynucleotide probe were isolated from G-CSF mobilized mononuclear peripheral blood. G-CSF-mobilized mononuclear cells may be obtained from any suitable human donor or are commercially available from BioWhittaker Inc. Walkersville, Md. USA. Mononuclear fractions can also be isolated from peripheral blood of patients treated with G-CSF using standard protocols.

According to one embodiment, in order to obtain maximum amounts of viable cells, labeling of cells may be performed with a DAZL polynucleotide probe following transfection according to well known protocols (for example Pederson T, Nucleic Acids Res. 29, 1013-1016, 2001). Following labeling, cells are subjected to cell sorting techniques to collect labeled cells. The isolated cells can be injected into severe combined immunodeficient (SCID) mice. Each injected mouse may form a teratoma that includes derivatives of all three germ layers, endoderm (e.g., gut epithelium), mesoderm (e.g., smooth and striated muscle) and ectoderm (e.g., neural epithelium and stratified squamous epithelium). This indicates in vivo pluripotency of the DAZL-labeled cells, similar to that expected of embryonic stem cells. The labeled cells isolated by this method may also be maintained and expanded in tissue culture in an undifferentiated state. According to various embodiments, these cells can be induced to differentiate into different cell types using state of the art methods (described in detail in the cell therapy section below).

The identification of the DAZL marker in embryonic stem cells was based in part on the hypothesis that germ stem cells and ES cells have common expression patterns that are distinct from somatic cells. Indeed, both, DAZL and Pum-2 are expressed in germ stem cells, and not in other somatic cells, and both are expressed also in ES (Moore et al. ibid). These genes may function with other genes in proliferation and maintenance of embryonic stem cells, in addition to their role in germ cell development. Genes and protein expressed in germ stem cells and in their progenitors, embryonic stem cells, and not expressed in differentiated somatic cells are used according to the present invention to isolate pluripotent or multipotent stem cells from blood, tissue and organs.

Prenatal Genetic Diagnosis

The DAZL gene can be used as a marker to isolate fetal cells from maternal peripheral blood for prenatal diagnostic purposes. A mononuclear fraction is isolated from peripheral blood of a pregnant woman according to well-known methods.

Using flow cytometry, cells can be analyzed and sorted on a flow sorter following staining with fluorescent-coupled DAZL monoclonal antibodies or by subjecting the cells to in situ hybridization with fluorescent-coupled DAZL oligonucleotide or nucleic acid probes. Under these conditions cells that have particular light scattering properties are also analyzed for the presence of fluorescence. When fluorescent-coupled antibodies are used, control experiments are performed using isotypically matched control monoclonal antibodies. When fluorescent-coupled oligonucleotide probes are used, controls consist of oligonucleotide sequences unrelated to mammalian sequences.

Once the fetal cells are isolated from the maternal blood, they can be cultured to increase the numbers of cells available for diagnosis (Fibach, et al., Blood, 73, 100, 1989) or can be analyzed directly.

Particular fetal proteins and/or nucleic acids can be selected or isolated from cells. If DNA amplification is to be carried out, DNA from the sorted samples is amplified for an appropriate number of cycles of denaturation and annealing (e.g., approximately 25-60). With proper modification of PCR conditions, more than one separate fetal gene can be amplified simultaneously. This technique, know as “multiplex” amplification, resulting in a mixture of products that contains amplified fetal DNA of interest, i.e., the DNA whose occurrence is to be detected and/or quantified (Chamberlin, et al., Prenat. Diagn., 9, 349-355, 1989).

The amplified fetal DNA of interest and other DNA sequences are separated, using known techniques. Subsequent analysis of amplified DNA also can be carried out using known techniques, such as: digestion with restriction endonuclease, ultraviolet light visualization of ethidium bromide stained agarose gels, DNA sequencing, or hybridization with allele specific oligonucleotide probes (Saiki, et al., Am. J. Hum. Genet. 43 (Suppl.), A35, 1988). The amplified mixture can also be separated on the basis of size and the resulting size-separated fetal DNA is contacted with an appropriate selected DNA probe or probes. Such analysis will determine whether polymorphic differences exist between the amplified “matemal” and “fetal” samples.

After the size-separated fetal DNA and the selected DNA probes have been maintained for sufficient time under appropriate conditions for hybridization of complementary DNA sequences to occur, resulting in production of fetal DNA/DNA probe complexes, detection of the complexes is carried out using known methods. For example, if the probe is labeled, the fetal DNA/labeled DNA probe complex is detected and/or quantified (e.g., by autoradiography, detection of the fluorescent label). The occurrence of fetal DNA associated with diseases or conditions can be detected and/or quantitated by the present method. In each case, an appropriate probe is used to detect the sequence of interest. For example, sequences from probes Stl4 (Oberle, et al, New Engl. J. Med., 312, 682-686, 1985), KM-19 (Gasparini, et al., Prenat. Diagnosis, 9, 349-355, 1989), or the deletion-prone exons for the Duchenne muscular dystrophy (DMD) gene (Chamberlain, et al., Nucleic Acids Res., 16, 11141-11156, 1988) are used as probes. Other conditions which can be diagnosed by the present method include Down's Syndrome, β-thalassemia (Cai, et al., Blood, 73, 372-374, 1989), sickle cell anemia (Saiki, et al., New. Engl. J. Med., 319, 537-541, 1988), phenylketonuria (DiLella, et al., Lancet, 1, 497-499, 1988) and Gaucher's disease (Theophilus, et al., Am. J. Hum. Genet., 45, 212-215, 1989).

The genetic abnormalities detected by the present invention can be deletions, additions, amplifications, translocations or rearrangements. For example, a deletion can be identified by detecting the absence of hybridizable binding of the probe to a target sequence. To detect a deletion of a genetic sequence, a population of probes are prepared that are complementary to the nucleic acid sequence that is present in a normal fetal cell but absent in an abnormal one. If the probes hybridize to the sequence in the cell being tested, then the sequence is detected and the cell is normal as to that sequence. If the probes fail to hybridize to cellular nucleic acid, then the sequence is not detected in that cell and the cell is designated as abnormal, provided that a control sequence, such as the X chromosome, is detected in the same cell.

Nucleotide additions can be identified by detecting binding of a labeled probe to a polynucleotide repeat segment of a chromosome. To detect an addition of a genetic sequence, such as an insertion in a chromosome or a karyotypic abnormality such as the trisomy of Chromosome 21 which indicates Down's Syndrome, a population of probes are prepared that are complementary to the genetic sequence in question. Continuing with the Down's Syndrome example, if the probes complementary to Chromosome 21 hybridize to three appearances of the Chromosome 21 sequence in the cell, then three occurrences of the Chromosome 21 sequence will be detected and indicate the Down's Syndrome trisomic condition. If the detection means is a fluorescent dye, for example, then three distinct points of fluorescence visible in each cell will indicate the trisomy condition. A translocation or rearrangement can be identified by several methods. For example, a labeled first probe may be bound to a marker region of a chromosome that has not translocated. A labeled second probe is then bound to a second region of the same chromosome (for a rearrangement) or a second chromosome (for a translocation) and subsequently binding of the first and second probes is detected. Alternatively, a translocation can be identified by first binding a labeled probe to a marker region of a polynucleotide section of a chromosome that translocates or rearranges, usually during metaphase. Subsequently, binding of the labeled probe is detected.

To detect a translocation, a marker for the chromosome in question is identified, and a population of probes is prepared that selectively hybridizes to it. Probes are marked with a detectable label, such as a dye that fluoresces at a particular wavelength. The sequence that translocates or rearranges in the abnormality being tested for is also identified, and second population of probes are prepared that identify it. The members of a second population of probes are marked with a distinguishably different label, such as a dye that fluoresces at a different wavelength from the first series of labeled probes. In situ hybridization is performed using both populations of probes, and the results of hybridization by each of the probe populations are compared. If the first and second labels are coincident on virtually all cell samples, no translocation has taken place. If the first label is found not to coincide with the second label on a significant fraction of samples, then a translocation or rearrangement has taken place (Speleman, Clinical Genetics, 41, 169-174, 1992).

“Nucleic acid probes” may be DNA or RNA fragments. DNA fragments can be prepared, for example, by digesting plasmid DNA, or by use of PCR, or synthesized by either the phosphoramidite method described by Beaucage and Carruthers (Tetrahedron Lett., 22, 1859-1862, 1981), or by the triester method according to Matteucci, et al. (J. Am. Chem. Soc., 103, 3185, 1981). A double stranded fragment may then be obtained, if desired, by annealing the chemically synthesized single strands together under appropriate conditions or by synthesizing the complementary strand using DNA polymerase with an appropriate primer sequence. Where a specific sequence for a nucleic acid probe is given, it is understood that the complementary strand is also identified and included. The complementary strand will work equally well in situations where the target is a double-stranded nucleic acid.

Oligonucleotide probes can be labeled at 5′ or 3′ overhanging ends as well as blunt-ended with terminal transferase and DIG-11-d UTP according to state of the art methods (Oligo nucleotide tailing Kit, described in Non-Radioactive Nucleic Acid Labeling and Detection protocols, Roche Diagnostics GmbH). The oligonuleotides probe produced by this method contain a tail which ranges in length from 10-100 nucleotides.

A variety of nucleic acid hybridization formats are known to those skilled in the art. For example, common formats include sandwich assays and competition or displacement assays. Hybridization techniques are generally described in “Nucleic Acid Hybridiziation, A Practical Approach,” Ed. Hames, and Higgins, IRL Press, 1985.

A kit for use in carrying out the present method of isolating and detecting fetal DNA of interest, such as a chromosomal abnormality associated with a disease or other condition, in a maternal blood sample can be produced. It includes, for example, a container for holding the reagents needed; the reagents and, optionally, a solid support for use in separating fetal nucleated cell/specific antibody complexes from other sample components or for removing maternal cells complexed with a specific antibody.

A typical kit can contain one or more DAZL antibodies, desirably bound to a solid support, to positively or negatively concentrate fetal cells within the specimen, probes specific for chromosome specific DNA sequences, means and instructions for performing fetal cell enrichment using density gradient centrifugation or flow cytometry, and optionally one or more reagents to prepare a density gradient that concentrates fetal cells. Such a kit would optionally provide reagents and materials for use in an automated system for the performance of any of the methods of the present invention.

Cell Therapy

A significant challenge to the use of stem cells for therapy is to control growth and differentiation into the particular type of tissue required for treatment of each patient.

U.S. Pat. No. 4,959,313 provides a particular enhancer sequence that causes expression of a flanking exogenous or recombinant gene from a promoter accompanying the gene that does not normally cause expression in undifferentiated cells. U.S. Pat. No. 5,639,618 proposes a method for isolating a lineage specific stem cell in vitro, in which a pluripotent embryonic stem cell is transfected with a construct in which a lineage-specific genetic element is linked to a reporter gene, culturing the cell under conditions where the cell differentiates, and then separation of cells expressing the reporter are separated from other cells.

U.S. Pat. No. 6,087,168 is directed to transdifferentiating epidermal cells into viable neurons useful for both cell therapy and gene therapy. Skin cells are transfected with a neurogenic transcription factor, and cultured in a medium containing an antisense oligonucleotide corresponding to a negative regulator of neuronal differentiation.

International Patent Publication WO 97/32025 proposes a method for engrafting drug resistant hematopoietic stem cells. The cells in the graft are augmented by a drug resistance gene (such as methotrexate resistant dihydrofolate reductase), under control of a promoter functional in stem cells. The cells are administered into a mammal, which is then treated with the drug to increase engraftment of transgenic cells relative to nontransgenic cells.

International Patent Publication WO 98/39427 refers to methods for expressing exogenous genes in differentiated cells such as skeletal tissue. Stem cells (e.g., from bone marrow) are contacted with a nucleic acid in which the gene is linked to an element that controls expression in differentiated cells. Exemplary is the rat osteocalcin promoter. International Patent Publication WO 99/10535 proposes a process for studying changes in gene expression in stem cells. A gene expression profile of a stem cell population is prepared, and then compared a gene expression profile of differentiated cells.

International Patent Publication WO 99/19469 refers to a method for growing pluripotent embryonic stem cells from the pig. A selectable marker gene is inserted into the cells so as to be regulated by a control or promoter sequence in the ES cells, exemplified by the porcine OCT-4 promoter.

International Patent Publication WO 00/15764 refers to propagation and derivation of embryonic stem cells. The cells are cultured in the presence of a compound that selectively inhibits propagation or survival of cells other than ES cells by inhibiting a signaling pathway essential for the differentiated cells to propagate. Exemplary are compounds that inhibit SHP-2, MEK, or the ras/MAPK cascade.

Differentiated cells of this invention can also be used for tissue reconstitution or regeneration in a human patient in need thereof. The cells are administered in a manner that permits them to graft to the intended tissue site and reconstitute or regenerate the functionally deficient area.

Differentiated cells of this invention can also be used for transplant therapy. For example, neural stem cells can be transplanted directly into parenchymal or intrathecal sites of the central nervous system, according to the disease being treated (U.S. Pat. No. 5,968,829). The efficacy of neural cell transplants can be assessed in a rat model for acutely injured spinal cord as described by McDonald et al. (Nat. Med. 5, 1410, 1999).

Certain neural progenitor differentiated cells of this invention are designed for treatment of acute or chronic damage to the nervous system. For example, excitotoxicity has been implicated in a variety of conditions including epilepsy, stroke, ischemia, Huntington's disease, Parkinson's disease and Alzheimer's disease. Certain differentiated cells of this invention may also be appropriate for treating dysmyelinating disorders, such as Pelizaeus-Merzbacher disease, multiple sclerosis, leukodystrophies, neuritis and neuropathies. Appropriate for these purposes are cell cultures enriched in oligodendrocytes or oligodendrocyte precursors to promote remyelination.

Hepatocytes and hepatocyte precursors prepared according to this invention can be assessed in animal models for ability to repair liver damage. One such example is damage caused by intraperitoneal injection of D-galactosamine (Dabeva et al., Am. J. Pathol. 143, 1606, 1993). Efficacy of treatment can be determined by immunohistochemical staining for liver cell markers, microscopic determination of whether canalicular structures form in growing tissue, and the ability of the treatment to restore synthesis of liver-specific proteins. Liver cells can be used in therapy by direct administration, or as part of a bioassist device that provides temporary liver function while the subject's liver tissue regenerates itself following fulminant hepatic failure.

The efficacy of cardiomyocytes prepared according to this invention can be assessed in animal models for cardiac cryoinjury, which causes 55% of the left ventricular wall tissue to become scar tissue without treatment (Li et al., Ann. Thorac. Surg. 62, 654, 1996; Sakai et al., Ann. Thorac. Surg. 8, 2074, 1999, Sakai et al., J. Thorac. Cardiovasc. Surg. 118, 715, 1999). Successful treatment will reduce the area of the scar, limit scar expansion, and improve heart function as determined by systolic, diastolic, and developed pressure. Cardiac injury can also be modeled using an embolization coil in the distal portion of the left anterior descending artery (Watanabe et al., Cell Transplant. 7, 239, 1998), and efficacy of treatment can be evaluated by histology and cardiac function. Cardiomyocyte preparations embodied in this invention can be used in therapy to regenerate cardiac muscle and treat insufficient cardiac function (U.S. Pat. No. 5,919,449 and WO 99/03973).

Gene Therapy

Gene therapy refers to the transfer and stable insertion of new genetic information into cells for the therapeutic treatment of diseases or disorders. The foreign gene is transferred into a cell that proliferates to spread the new gene throughout the cell population. Thus stem cells, or pluripotent progenitor cells, are usually the target of gene transfer, since they are proliferative cells that produce various progeny lineages which will potentially express the foreign gene.

Stem cells selected using the marker DAZL may be used in gene therapy for the treatment of a variety of diseases, particularly genetic diseases. Genetic diseases associated with hematopoietic cells may be treated by genetic modification of autologous or allogeneic stem cells to correct the genetic defect. For example, diseases including, but not limited to, β-thalassemia, sickle cell anemia, adenosine deaminase deficiency, recombinase deficiency, recombinase regulatory gene deficiency, etc. may be corrected by introduction of a wild-type gene into the selected DAZL cells, either by homologous or random recombination. Other indications of gene therapy are introduction of drug resistance genes to enable normal stem cells to have an advantage and be subject to selective pressure during chemotherapy. Diseases other than those associated with hematopoietic cells may also be treated by genetic modification, where the disease is related to the lack of a particular secreted product including, but not limited to, hormones, enzymes, interferons, growth factors, or the like. By employing an appropriate regulatory initiation region, inducible production of the deficient protein may be achieved, so that production of the protein will parallel natural production, even though production will be in a different cell type from the cell type that normally produces such protein. It is also possible to insert a ribozyme, antisense or other message to inhibit particular gene products or susceptibility to diseases, particularly hematolymphotropic diseases.

Alternatively, one may wish to remove a particular variable region of a T-cell receptor from the T-cell repertoire. By employing homologous recombination, or antisense or ribozyme sequence which prevents expression, the expression of the particular T-cell receptor may be inhibited. For hematotrophic pathogens, such as HIV, HTLV-I and II, etc. the stem cells could be genetically modified to introduce an antisense sequence or ribozyme which would prevents the proliferation of the pathogen in the stem cell or cells differentiated from the stem cells. Methods for recombination in mammalian cells may be found in Molecular Cloning, A Laboratory Manual (1989) Sambrook, Fritsch and Maniatis, Cold Spring Harbor, N.Y.

Optionally, the progenitor cells obtained using the method of the present invention can be manipulated to express desired gene products. Gene therapy can be used to either modify a cell to replace a gene product, to facilitate regeneration of tissue, to treat disease, or to improve survival of the cells following implantation into a patient (i.e. prevent rejection). In this embodiment, the progenitor cells are transfected prior to expansion and differentiation. Techniques for transfecting cells are known in the art.

A skilled artisan could envision a multitude of genes which would convey beneficial properties to the transfected cell or, more indirectly, to the recipient patient/animal. The added gene may ultimately remain in the recipient cell and all its progeny, or may only remain transiently, depending on the embodiment. For example, genes encoding angiogenic factors could be transfected into progenitor cells isolated from smooth muscle. Such genes would be useful for inducing collateral blood vessel formation as the smooth muscle tissue is regenerated. It some situations, it may be desirable to transfect the cell with more than one gene.

In some instances, it is desirable to have the gene product secreted. In such cases, the gene product preferably contains a secretory signal sequence that facilitates secretion of the protein. For example, if the desired gene product is an angiogenic protein, a skilled artisan could either select an angiogenic protein with a native signal sequence, e.g. VEGF, or can modify the gene product to contain such a sequence using routine genetic manipulation (Nabel J. G. et al., Thromb Haemost. 70, 202-203, 1993). The desired gene can be transfected into the cell using a variety of techniques. Preferably, the gene is transfected into the cell using an expression vector. Suitable expression vectors include plasmid vectors, viral vectors (such as replication defective retroviral vectors, herpes virus, adenovirus, adenovirus associated virus, and lentivirus), and non-viral vectors (such as liposomes or receptor ligands).

The desired gene is usually linked to its own promoter or to a foreign promoter which, in either case, mediates transcription of the gene product. Promoters are chosen based on their ability to drive expression in restricted or in general tissue types, or on the level of expression they promote, or how they respond to added chemicals, drugs or hormones. Other genetic regulatory sequences that alter expression of a gene may be co-transfected. In some embodiments, the host cell DNA may provide the promoter and/or additional regulatory sequences. Cells containing the gene may then be selected for by culturing the cells in the presence of the toxic compound. Methods of targeting genes in mammalian cells are well known to those of skill in the art (U.S. Pat. Nos. 5,830,698; 5,789,215; 5,721,367 and 5,612,205).

The methods of the present invention may used to isolate and enrich stem cells or progenitors cells that are capable of homologous recombination and, therefore, subject to gene targeting technology. Most studies in gene therapy have focused on the use of hematopoietic stem cells. High efficiency gene transfer systems for hematopoietic progenitor cell transformation have been investigated for use (Morrow, J. F., Ann. N.Y. Acad. Sci. 265, 13, 1976; Dick, J. E., et al., Trends in Genetics 2, 165, 1986). Reports on the development of viral vector systems indicate a higher efficiency of transformation than DNA-mediated gene transfer procedures (e.g., CaPO.sub.4 precipitation and DEAE dextran) and show the capability of integrating transferred genes stably in a wide variety of cell types. Recombinant retrovirus vectors have been widely used experimentally to transduce hematopoietic stem and progenitor cells. Genes that have been successfully expressed in mice after transfer by retrovirus vectors include human hypoxanthine phosphoribosyl transferase (Miller, A., et al. Science 255, 630, 1984). Bacterial genes have also been transferred into mammalian cells, in the form of bacterial drug resistance gene transfers in experimental models. The transformation of hematopoietic progenitor cells to drug resistance by eukaryotic virus vectors has been accomplished with recombinant retrovirus-based vector systems (Hock, R. A. and Miller, A. D. Nature 320, 275-277, 1986; Dick, J. E., et al. Cell 42, 71-79, 1985; Eglitis, M., et al., Science 230, 1395-1398, 1985). Recently, adeno-associated virus vectors have been used successfully to transduce mammalian cell lines to neomycin resistance (Tratschin, J. D. et al. Mol. Cell. Biol. 5, 3251, 1985). Other viral vector systems that have been investigated for use in gene transfer include papovaviruses and vaccinia viruses (see Cline, M. J. Pharmac. Ther. 29, 69-92, 1985).

Other methods of gene transfer include microinjection, electroporation, liposomes, chromosome transfer, and transfection techniques such as calcium-precipitation transfection technique to transfer a methotrexate-resistant dihydrofolate reductase (DHFR) or the herpes simplex virus thymidine kinase gene, and a human globin gene into murine hematopoietic stem cells. In vivo expression of the DHFR and thymidine kinase genes in stem cell progeny was demonstrated (Salser, W., et al. in Organization and Expression of Globin Genes, Alan R. Liss, Inc., New York, pp. 313-334, 1981).

Gene therapy has also been investigated in murine models with the goal of enzyme replacement therapy. Normal stem cells from a donor mouse have been used to reconstitute the hematopoietic cell system of mice lacking beta-glucuronidase (Yatziv, S. et al. J. Lab. Clin. Med. 90, 792-797, 1982). By this way, a native gene was being supplied and no recombinant stem cells (or gene transfer techniques) were needed.

The present invention further encompasses methods for obtaining compositions of cells which are highly enriched in stem cells. The method comprises incubating the compositions described above under conditions suitable for regeneration of stem cells. Compositions comprising the original stem cells and/or the regenerated stem cells are obtained thereby. Such a composition has utility in reconstituting human hematopoietic systems and in studying various parameters of hematopoietic cells as described above.

The invention also encompasses methods of use of the selected DAZL stem cell populations. The subject cell compositions may find use in any method known in the art. Since the cells are naive, they can be used to fully reconstitute an immunocompromised host such as an irradiated host or a host subject to chemotherapy; or as a source of cells for specific lineages, by providing for their maturation, proliferation and differentiation into one or more selected lineages by employing a variety of factors, including, but not limited to, erythropoietin, colony stimulating factors, e.g., GM-CSF, G-CSF, or M-CSF, interleukins, e.g., IL-1, -2, -3, -4, -5, -6, -7, -8, etc., or the like, or stromal cells associated with the stem cells becoming committed to a particular lineage, or with their proliferation, maturation and differentiation. The selected DAZL stem cells may also be used in the isolation and evaluation of factors associated with the differentiation and maturation of hematopoietic cells. Thus, the selected DAZL stem cells may be used in assays to determine the activity of media, such as conditioned media, evaluate fluids for cell growth activity, involvement with dedication of particular lineages, or the like.

Separation Methods

Separation may be performed according to various physical properties, such as fluorescent properties or other optical properties, magnetic properties, density, electrical properties, etc. Cell types can be isolated by a variety of means including fluorescence activated cell sorting (FACS), protein-conjugated magnetic bead separation, morphologic criteria, specific gene expression patterns (using RT-PCR), or specific antibody staining.

The use of separation techniques include, but are not limited to, those based on differences in physical (density gradient centrifugation and counter-flow centrifugal elutriation), cell surface (lectin and antibody affinity), and vital staining properties (mitochondria-binding dye rhol23 and DNA-binding dye Hoechst 33342).

Cells may be selected based on light-scatter properties as well as their expression of various cell surface antigens. The purified stem cells have low side scatter and low to medium forward scatter profiles by FACS analysis. Cytospin preparations show the enriched stem cells to have a size between mature lymphoid cells and mature granulocytes.

Various techniques can be employed to separate the cells by initially removing cells of dedicated lineage. Monoclonal antibodies are particularly useful. The antibodies can be attached to a solid support to allow for crude separation. The separation techniques employed should maximize the retention of viability of the fraction to be collected.

The separation techniques employed should maximize the retention of viability of the fraction to be collected. Various techniques of different efficacy may be employed to obtain “relatively crude” separations. Such separations are where up to 10%, usually not more than about 5%, preferably not more than about 1%, of the total cells present are undesired cells that remain with the cell population to be retained. The particular technique employed will depend upon efficiency of separation, associated cytotoxicity, ease and speed of performance, and necessity for sophisticated equipment and/or technical skill.

Procedures for separation may include magnetic separation, using antibody-coated magnetic beads, affinity chromatography, cytotoxic agents joined to a monoclonal antibody or used in conjunction with a monoclonal antibody, e.g., complement and cytotoxins, and “panning” with antibody attached to a solid matrix, e.g., plate, or other convenient technique.

Techniques providing accurate separation include fluorescence activated cell sorters, which can have varying degrees of sophistication, e.g., a plurality of color channels, low angle and obtuse light scattering detecting channels, impedance channels, etc.

Other techniques for positive selection may be employed, which permit accurate separation, such as affinity columns, and the like. The method should permit the removal to a residual amount of less than about 20%, preferably less than about 5%, of the non-target cell populations.

The antibodies may be conjugated with markers, such as magnetic beads, which allow for direct separation, biotin, which can be removed with avidin or streptavidin bound to a support, fluorochromes, which can be used with a fluorescence activated cell sorter, or the like, to allow for ease of separation of the particular cell type. Any technique may be employed which is not unduly detrimental to the viability of the remaining cells.

Conveniently, after substantial enrichment of the cells lacking the DAZL marker, generally by at least about 50%, preferably at least about 70%, the cells may now be separated by a fluorescence activated cell sorter (FACS) or other methodology having high specificity. Multi-color analyses may be employed, with the FACS which is particularly convenient. The cells may be separated on the basis of the level of staining for the particular antigens.

While it is believed that the particular order of separation is not critical to this invention, the order indicated is preferred. Preferably, cells are initially separated by a coarse separation, followed by a fine separation, with positive selection of one or more markers associated with the stem cells and negative selection for markers associated with lineage committed cells.

Cryopreservation

The freezing of cells is ordinarily destructive. On cooling, water within the cell freezes. Injury then occurs by osmotic effects on the cell membrane, cell dehydration, solute concentration, and ice crystal formation. As ice forms outside the cell, available water is removed from solution and withdrawn from the cell, causing osmotic dehydration and raised solute concentration which eventually destroys the cell. These injurious effects can be circumvented by (a) use of a cryoprotective agent, (b) control of the freezing rate, and (c) storage at a temperature sufficiently low to minimize degradative reactions.

Cryoprotective agents which can be used include but are not limited to dimethyl sulfoxide (DMSO), glycerol, polyvinylpyrrolidine, polyethylene glycol, albumin, dextran, sucrose, ethylene glycol, i-erythritol, D-ribitol, D-mannitol, D-sorbitol, i-inositol, D-lactose, choline chloride, amino acids, methanol, acetamide, glycerol monoacetate, and inorganic salts.

In a preferred embodiment, DMSO is used, a liquid which is nontoxic to cells in low concentration. Being a small molecule, DMSO freely permeates the cell and protects intracellular organelles by combining with water to modify its freezability and prevent damage from ice formation. Addition of plasma (e.g., to a concentration of 20-25%) can augment the protective effect of DMSO. After addition of DMSO, cells should be kept at 0° C. until freezing, since DMSO concentrations of about 1% are toxic at temperatures above 4° C.

A controlled slow cooling rate is critical. Different cryoprotective agents and different cell types have different optimal cooling rates (Lewis, J. P., et al. Transfusion 7, 17-32, 1967). The heat of fusion phase where water turns to ice should be minimal. The cooling procedure can be carried out by use of, e.g., a programmable freezing device or a methanol bath procedure. Programmable freezing apparatuses allow determination of optimal cooling rates and facilitate standard reproducible cooling. Programmable controlled-rate freezers such as Cryomed or Planar permit tuning of the freezing regimen to the desired cooling rate curve. For example, for marrow cells in 10% DMSO and 20% plasma, the optimal rate is 1 to 3° C./minute from 0° C. to −80° C. In a preferred embodiment, this cooling rate can be used for the neonatal cells of the invention. The container holding the cells must be stable at cryogenic temperatures and allow for rapid heat transfer for effective control of both freezing and thawing. Sealed plastic vials (e.g., Nunc, Wheaton cryules) or glass ampules can be used for multiple small amounts (1-2 ml), while larger volumes (100-200 ml) can be frozen in polyolefin bags (e.g., Delmed) held between metal plates for better heat transfer during cooling. (Bags of bone marrow cells have been successfully frozen by placing them in −80° C. freezers which, fortuitously, gives a cooling rate of approximately 3° C./minute).

In an alternative embodiment, the methanol bath method of cooling can be used. The methanol bath method is well-suited to routine cryopreservation of multiple small items on a large scale. The method does not require manual control of the freezing rate nor a recorder to monitor the rate. In a preferred aspect, DMSO-treated cells are precooled on ice and transferred to a tray containing chilled methanol which is placed, in turn, in a mechanical refrigerator (e.g., Harris or Revco) at −80° C. Thermocouple measurements of the methanol bath and the samples indicate the desired cooling rate of 1 to 3° C./minute. After at least two hours, the specimens have-reached a temperature of −8° C. and can be placed directly into liquid nitrogen (−196° C.) for permanent storage.

After thorough freezing, cells can be rapidly transferred to a long-term cryogenic storage vessel. In a preferred embodiment, samples can be cryogenically stored in liquid nitrogen (−196° C.) or its vapor (−165° C.). Such storage is greatly facilitated by the availability of highly efficient liquid nitrogen refrigerators, which resemble large Thermos containers with an extremely low vacuum and internal super insulation, such that heat leakage and nitrogen losses are kept to an absolute minimum.

Considerations and procedures for the manipulation, cryopreservation, and long-term storage of hematopoietic stem cells, particularly from bone marrow or peripheral blood, are largely applicable to the neonatal and fetal stem cells of the invention (Gorin, N.C. Clinics In Haematology 15, 19-48, 1986)

Other methods of cryopreservation of viable cells, or modifications thereof, are available and envisioned for use (e.g., cold metal-mirror techniques; U.S. Pat. No. 4,199,022; U.S. Pat. No. 3,753,357; U.S. Pat. No. 4,559,298). U.S. Pat. No. 6,310,195 discloses a method for preservation of pluripotent progenitor cells, as well as totipotent progenitor cells based on a use of a specific protein. In a preferred case, the protein can preserve hematopoietic progenitor cells, but progenitor cells from other tissues can also be preserved, including nerve, muscle, skin, gut, bone, kidney, liver, pancreas, or thymus progenitor cells.

Frozen cells are preferably thawed quickly (e.g., in a water bath maintained at 37-41° C.) and chilled immediately upon thawing. In particular, the vial containing the frozen cells can be immersed up to its neck in a warm water bath; gentle rotation will ensure mixing of the cell suspension as it thaws and increase heat transfer from the warm water to the internal ice mass. As soon as the ice has completely melted, the vial can be immediately placed in ice.

In Vitro Cultures of Hematopoietic Stem and Progenitor Cells

An optional procedure (either before or after cryopreservation) is to expand the hematopoietic stem and progenitor cells in vitro. However, care should be taken to ensure that growth in vitro does not result in the production of differentiated progeny cells at the expense of multipotent stem and progenitor cells which are therapeutically necessary for hematopoietic reconstitution. Various protocols have been described for the growth in vitro of cord blood or bone marrow cells, and it is envisioned that such procedures, or modifications thereof, may be employed (Dexter, T. M. et al. J. Cell. Physiol. 91, 335, 1977; Witlock, C. A. and Witte, O. N. Proc. Natl. Acad. Sci. U.S.A. 79, 3608-3612, 1982). Various factors can also be tested for use in stimulation of proliferation in vitro, including but not limited to interleukin-3 (IL-3), granulocyte-macrophage (GM)-colony stimulating factor (CSF), IL-1 (hemopoietin-1), IL-4 (B cell growth factor), IL-6, alone or in combination.

The following examples are intended to illustrate how to make and use the compounds and methods of this invention and are in no way to be construed as a limitation. Although the invention will now be described in conjunction with specific embodiments thereof, it is evident that many modifications and variations will be apparent to those skilled in the art. Accordingly, it is intended to embrace all such modifications and variations that fall within the spirit and broad scope of the appended claims.

### EXAMPLES

Experiments were performed to demonstrate that the DAZL gene product, and products of other genes in its class, can be used to identify “embryonic-like” stem cells in blood or other sources.

DAZL expression was first studied by reverse transcriptase PCR (RT-PCR). It was found that DAZL is transcribed in embryonic stem cells, but is not transcribed in other cell-lines or tissues. Then DAZL gene expression in blood stem cells was checked. Since there are only a few stem cells in blood circulation, the expression of DAZL gene was studied first in bone marrow by immunofluorescence using antibodies specific to the DAZL protein. Immunofluorescence results demonstrated the expression of DAZL in a few cells, presumably stem cells, while no expression was observed in differentiated cells.

To characterize the cell fraction labeled with DAZL antibodies, the expression of DAZL was studied in peripheral blood isolated from patients treated with G-CSF to accelerate stem cell release to blood circulation. DAZL expression in G-CSF mobilized mononuclear cells has been identified by RT-PCR.

FACS analysis of DAZL-labeled G-CSF mobilized mononuclear cell supports the observation that DAZL expresses in a small subset of mononuclear cells. A small subset (about 0.1%) of cells demonstrating a significantly higher fluorescent than control was seen in DAZL labeled cells. Such a subset of cells was also observed by fluorescent microscopy.

Immunofluorescence of G-CSF mobilized mononuclear cells demonstrates DAZL expression in a few mononuclear cells. It is estimated that about 0.1% of the mononuclear cells are labeled with DAZL antibodies.

### EXAMPLE 1

### Study of DAZL Expression in Embryonic Stem Cells and Other Tissues and Cell Lines by Reverse Transcriptase PCR

As primordial germ cells and embryonic stem cells demonstrate similar pluripotent characteristics, it was examined whether these cell-lines possess high similarity in gene expression. The DAZ family genes, including DAZL are unique in their expression in very early developmental stages of germ cells, presumable primordial germ cells. A functional role of these genes in primordial germ cells development has been suggested (Brekhman et al., Mol Human Reprod 6, 465-468, 2000). The DAZL gene is autosomal, expresses in male and female germ cells, and therefore was tested for expression in embryonic stem cells.

RNAs were extracted from ES cells, a MEF cell line, FHs173 cell line, human kidney and spleen using Trizol® reagent (Gibco BRL, Grand Island, N.Y.) according to the manufacturer's instruction. These RNAs were used to amplify a 357 bp fragment of human DAZL (cDNA cycle kit, Invitrogen) using a forward primer: 3′ AGCCACGTCCTTTGGTTTT 5′ (SEQ ID NO:4) and a reverse primer: 3′ GTGGGCCATTTCCAGAGGG 5′ (SEQ ID NO:5).

cell-lines or tissues. Then DAZL gene expression in blood stem cells was checked. Since there are only a few stem cells in blood circulation, the expression of DAZL gene was studied first in bone marrow by immunofluorescence using antibodies specific to the DAZL protein. Immunofluorescence results demonstrated the expression of DAZL in a few cells, presumably stem cells, while no expression was observed in differentiated cells.

To characterize the cell fraction labeled with DAZL antibodies, the expression of DAZL was studied in peripheral blood isolated from patients treated with G-CSF to accelerate stem cell release to blood circulation. DAZL expression in G-CSF mobilized mononuclear cells has been identified by RT-PCR.

FACS analysis of DAZL-labeled G-CSF mobilized mononuclear cell supports the observation that DAZL expresses in a small subset of mononuclear cells. A small subset (about 0.1%) of cells demonstrating a significantly higher fluorescent than control was seen in DAZL labeled cells. Such a subset of cells was also observed by fluorescent microscopy.

Immunofluorescence of G-CSF mobilized mononuclear cells demonstrates DAZL expression in a few mononuclear cells. It is estimated that about 0.1% of the mononuclear cells are labeled with DAZL antibodies.

### EXAMPLE 1

### Study of DAZL Expression in Embryonic Stem Cells and Other Tissues and Cell Lines by Reverse Transcriptase PCR

As primordial germ cells and embryonic stem cells demonstrate similar pluripotent characteristics, it was examined whether these cell-lines possess high similarity in gene expression. The DAZ family genes, including DAZL are unique in their expression in very early developmental stages of germ cells, presumable primordial germ cells. A functional role of these genes in primordial germ cells development has been suggested (Brekhman et al., Mol Human Reprod 6, 465-468, 2000). The DAZL gene is autosomal, expresses in male and female germ cells, and therefore was tested for expression in embryonic stem cells.

RNAs were extracted from ES cells, a MEF cell line, FHs 173 cell line, human kidney and spleen using Trizol® reagent (Gibco BRL, Grand Island, N.Y.) according to the manufacturer's instruction. These RNAs were used to amplify a 357 bp fragment of human DAZL (cDNA cycle kit, Invitrogen) using a forward primer: 3′ AGCCACGTCCTTTGGTTTT 5′ (SEQ ID NO:4) and a reverse primer: 3′ GTGGGCCATTTCCAGAGGG 5′ (SEQ ID NO:5).

The gene encoding Beta-actin was used as a reference reaction using a forward primer: 3′ CGACGAGGCCCAGAGCAAGAGAGG 5′ (SEQ ID NO:6) and a reverse primer: 3′ CGTCAGGCAGCTCATAGCTCTTCTCCAGGG 5′ (SEQ ID NO:7).

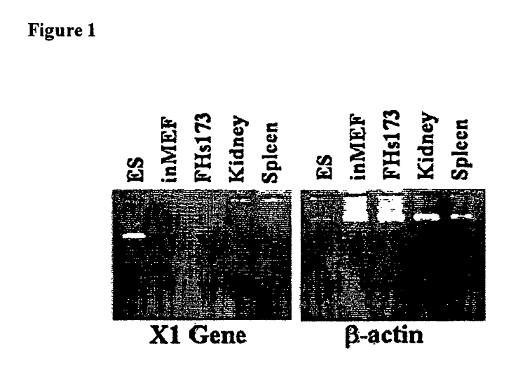

It was found that DAZL is transcribed in embryonic stem cells (FIG. 1), testis and testicular Teratomas cell (not shown), but is not transcribed in other embryonic and adult cell-lines or tissues. The results support previous reports indicating that DAZL expression is limited to the human testes and ovaries and is not expressed in other organs. The DAZ/DAZL associated gene, pum-2 was recently shown to be expressed in embryonic stem cells in a similar manner as DAZL gene (Moore et al. ibid).

### EXAMPLE 2

### Use of the DAZL to Identify “Embryonic-Like” Stem Cells in Adult Organs

In order to examine whether DAZL is expressed in stem cells of the blood system, DAZL expression in bone marrow samples was examined. Normally there are only a small fraction of stem cells in blood circulation, the expression of DAZL in bone marrow cells was analyzed by immunofluorescence using DAZL antibodies. These antibodies were raised against the C-terminal 183 amino acids of the mouse Dazl and affinity purified with DAZL peptide (Ruggui et al. Nature 389, 73-77, 1997; Ruggiu et al., J. Androl 22, 470-477, 2000).

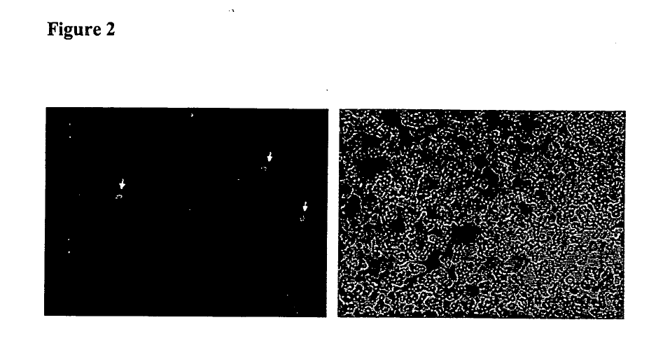

Bone marrow cells were reacted with DAZL antibodies following fixation with 2% paraformaldehyde (PFA) onto microscopic slides. Slides were incubated for 30 min with 0.05% NP-40 in PBS containing 10% fetal calf serum (FCS), washed and then incubated for two hours with 1/50 dilution of DAZL antibodies. Controls were incubated with preimmune sera. Next, slides were washed with PBS and stained with 1/500 dilution of second antibodies anti-rabbit IgG conjugated with FITC. Slides were washed, mounted and analyzed using an Olympus fluorescent microscope. Immunofluorescence results demonstrated the expression of DAZL in a small fraction of undifferentiated blood cells, presumably stem cells, while no expression was observed in red blood cells. As can be seen in FIG. 2, three cells were labeled with DAZL antibodies out of an estimated 500 cells.

### EXAMPLE 3

### Existence of DAZL in Adult Peripheral Blood

As DAZL expression could not be detected in normal peripheral blood, its expression in mobilized peripheral blood mononuclear cells was examined. The peripheral blood mononuclear cells contain elevated numbers of progenitor cells mobilized from bone marrow by administration of Neupogen (G-CSF). Such G-CSF mobilized mononuclear cells from normal donors, containing about 0.91% CD34+cells, were obtained from BioWhittaker Inc. Walkersville, Md. USA.

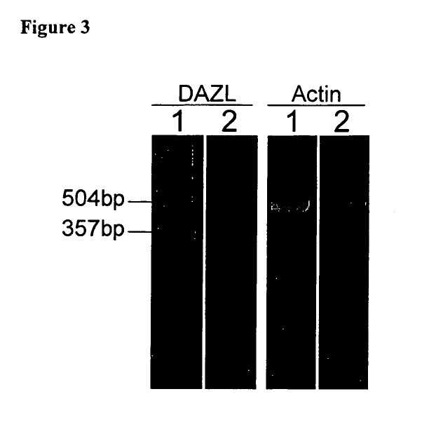

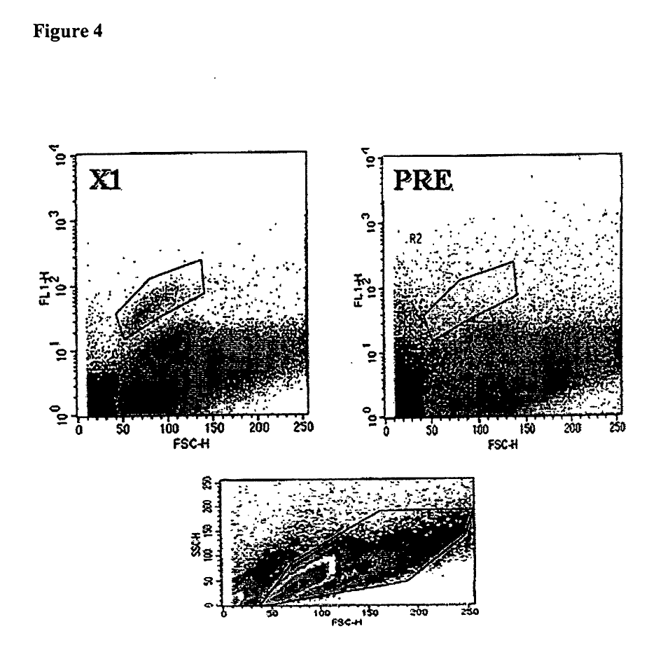

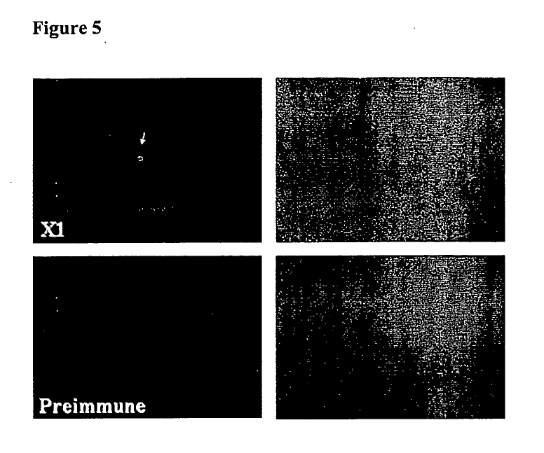

RNA was extracted from mononuclear blood cells and the DAZL transcript was amplified by RT-PCR. As can be seen in FIG. 3, DAZL transcript is identified in G-CSF mobilized mononuclear cells. Immunofluorescent and FACS analysis of DAZL labeled G-CSF mobilized mononuclear cells support the observation that DAZL expresses in a small subset of mononuclear cells, presumably stem cells. As can be seen in FIG. 4, immunofluorescent analysis by FACS demonstrated a significant labeling in a subset comprising about 0.1% of total cells. This subset of cells did not stain with preimmune sera samples. Such subset of cells was also observed by a fluorescent microscope. Immunofluorescence of G-CSF mobilized mononuclear cells demonstrates DAZL expression in a few mononuclear cells. It is estimated that about 0.1% of the mononuclear cells labeled with DAZL antibodies. The FACS results were supported by microscopic observation of immunofluorescent labeled cells as shown in FIG. 5 that demonstrates the ratio of DAZL expressing cells in the mononuclear cells. As can be seen, one strongly labeled cell is identified out of many that do not labeled.

### EXAMPLE 4

### Production of Monoclonal Antibodies Specific to DAZL

To improve reactions and to lower background, monoclonal antibodies specific for DAZL somatic protein are produced. A short protein, comprising 150 amino acids of the C-terminal of DAZL (SEQ ID NO:3) is injected into mice. Monoclonal antibodies are produced by standard techniques which are well known in the art (Kozbar et al., Immunology Today 4, 72, 1983; Cole et al., Monoclonal Antibodies and Cancer Therapy, Alan R. Liss, Inc., pp. 77-96, 1985). Antibodies selective for DAZL in somatic stem cells, are preferably chosen over those which detect DAZL in germ cells, based on the assumption that different isoforms of DAZL are expressed in somatic cells as compared to germ cells. This is performed by selection of hybridomas that express antibodies that exhibit strong immunofluorescent signals and western blot with the somatic stem cell protein.

| C-terminal 150 amino acid fragment of DAZL: | |
|---|---|
| SEQ ID NO:3 | |
| 1 | PNTETYMQPT TTMNPITQYV QAYPTYPNSP VQVITGYQLP VYNYQMPPQW |
| 51 | PVGEQRSYVV PPAYSAVNYH CNEVDPGAEV VPNECSVHEA TPPSGNGPQK |
| 101 | KSVDRSIQTV VSCLFNPENR LRNSVVTQDD YFKDKRVHHF RRSRAMLKSV |

Sample 5: Isolation of Mononuclear blood cells.

Mononuclear cells are isolated from venous blood of healthy donors treated with G-CSF (Neupogen, Amgen, Inc, 7.5 μg/kg body weight/day) or from pregnant women at 18 weeks of gestation. Mononuclear cells are prepared by centrifugation in a density cell separation medium. Briefly, blood (about 43 ml) is collected into a 60 ml syringe containing 10 ml 6% dextran and 7 ml citrate/citric acid, mixed and sedimented for 30 min at room temperature. The serum/white blood cells fraction is transferred to 50 ml tube, underlayed with 12 ml RT Histopaque 1077 and spin at 2500 rpm for 30 min. The serum above the band is removed and then the mononuclear band (with some Ficoll) is taken and transfered into two 15 ml tubes. Cold media (1×HBSS (10×:50 ml), 10 mM Hepes (1M: 5 ml), 5 mM EDTA (0.5M: 5 ml), 2.5% FBS (12.5 ml) in 500 ml) is added to a final volume of 15 ml, and cells are centrifuged at 1800 rpm, 4° C. for 5 min. Pellets are pooled into one 15 ml tube and cold media is added. Mononuclear cells are washed three times (1400 rpm, 4° C., for 5 min) to remove platelets.

Sample 6: Isolation of Fetal Cells from Maternal Blood.

Mononuclear cells isolated from a pregnant woman are washed (1400 RPM, 5 min, 4° C.) once in phosphate-buffered saline (PBS). The pellet is resuspended in 10 ml cold PBS to a concentration of about 5×10⁶ cells/ml and then an equal volume of 4% formaldehyde solution is added. The mixture is immediately vortexed briefly and incubated for 30 min at 4° C. Cells are centrifuged, resuspended in 10 ml PBS with 2% newborn calf serum and 0.2% Tween 20 and are incubated for 30 min in 37° C. water bath. Cells are then centrifuged and pellets are resuspended in 500 μl PBS with 1% newborn calf serum and a 1/100 dilution of DAZL antibody, vortexed briefly and incubated for 1 hour at 4° C. Cells are washed twice with 0.2% Tween 20 in PBS. Pellets are resuspended in 500 μl PBS with 1/500 dilution of Cy2-conjugated affinipure goat anti rabbit IgG (Jackson ImmunoResearch Laboratories) and incubated for 15 min at 4° C. in the dark. Cells are washed again twice in PBS and kept in dark before analysis. Cells are analyzed by fluorescent microscope with a micromanipulator system (TDU5000 Micromanipulator, Integra UK) such as used for micromanipulations of embryos. Labeled fluorescent cells are isolated by micropipette. About 50-100 cells can be isolated from one blood sample.

For genetic analyses by PCR, pools of cells are inserted into each PCR tube containing 10 μl sterile water (strips of 8 tubes of 200 μl volume are used). The tubes are then frozen and thawed three times (thaw at 100° C. for 1 min and freeze in liquid nitrogen for 1 min) and kept in −80 until analysis. For chromosomal analysis by FISH (fluorescent in-situ hybridization), single cells are placed on prepared glass slides and fixed (Munneet al. 1998). Briefly, cells are incubated in 2×SSC at 37° C. for 30 min followed by dehydration in chilled alcohol gradients for 1 minute each. After wiping the excess alcohol slides are prepared for hybridization (such as by Vysis probe cocktail).

It will be appreciated that many possible alternatives will be apparent within the scope of the present invention which is not to be limited by the specific embodiments exemplified herein but rather by the following claims.

1. A method for identifying, characterizing, selecting or isolating pluripotent or multipotent stem cells in a population of mammalian cells which comprises using an embryonic stem cell marker that exhibits selective expression in primordial germ cells or germ stem cells and an absence of expression in differentiated somatic cells to assist in identifying, characterizing, selecting or isolating the pluripotent or multipotent stem cells.

2. The method of claim 1 wherein the embryonic stem cell marker is used to identify the pluripotent or multipotent stem cells and the method further comprises selecting the identified pluripotent or multipotent stem cells for collection.

3. The method of claim 2 which further comprises separating the selected pluripotent or multipotent stem cells from the population of mammalian cells.

4. The method of claim 3 which further comprises isolating the separated pluripotent or multipotent stem cells.

5. The method of claim 1 wherein the cell population is selected from peripheral blood, cord blood, body fluids, tissue samples, tissue cultures, bone marrow cells, organ samples, organ cultures, cell lines and cell cultures.

6. The method of claim 1 wherein the stem cells are adult stem cells, embryonic stem cells or stem cells of fetal origin.

7. The method of claim 1 wherein the stem cells are of human fetal origin within a maternal cell population.

8. The method of claim 1 wherein the marker comprises at least one of: (i) a DAZL gene transcript having SEQ ID NO: 1; (ii) a polynucleotide that hybridizes to (i); (iii) a DAZL protein having SEQ ID NO: 2; (iv) a peptide fragment of (iii), or (v) a protein having at least one epitope of (iii).

9. The method of claim 1 wherein expression of the marker is tested using a reagent selected from a polyclonal antibody, a monoclonal antibody, an antibody fragment, a polynucleotide probe, or an oligonucleotide probe.

10. The method of claim 9 wherein the reagent comprises a DAZL polynucleotide probe or a polynucleotide having at least 70% homology thereto, at least 80% homology thereto, or at least 90% homology thereto.

11. The method of claim 9 wherein the reagent comprises at least the antigen binding portion of an antibody specific to at least one epitope of a DAZL protein.

12. A method for identification, selection or characterization of pluripotent or multipotent embryonic stem cells from mammalian fluids or tissues which comprises obtaining an antibody specific to at least one epitope of the DAZL protein and contacting the antibody with the stem cells to identify, select or characterize such cells.

13. The method of claim 12 wherein the antibody comprises the antigen binding portion of an immunoglobulin specifically recognizing and binding a polypeptide having at least 70% homology to SEQ ID NO: 2, the antigen binding portion of an immunoglobulin specifically recognizing and binding a polypeptide having at least 80% homology to SEQ ID NO: 2, the antigen binding portion of an immunoglobulin specifically recognizing and binding a polypeptide having at least 90% homology to SEQ ID NO: 2, the antigen binding portion of an immunoglobulin specifically recognizing and binding a polypeptide having at least 70% homology to SEQ ID NO: 3, the antigen binding portion of an immunoglobulin specifically recognizing and binding a polypeptide having at least 80% homology to SEQ ID NO: 3, the antigen binding portion of an immunoglobulin specifically recognizing and binding a polypeptide having at least 90% homology to SEQ ID NO: 3, or a peptide fragment retaining antigenic specificity of at least one epitope of DAZL.

14. Mammalian stem cells isolated by the method of claim 1.

15. Mammalian stem cells isolated by the method of claim 12.

16. A method for diagnosis of prenatal genetic disorders which comprises identifying, selecting or characterizing embryonic stem cells from mammalian tissue or fluid of a pregnant woman by the method of claim 1 and separating or isolating the identified stem cells for use in the diagnosis of prenatal genetic disorders.

17. A method for diagnosis of prenatal genetic disorders which comprises identifying, selecting or characterizing embryonic stem cells from mammalian tissue or fluid of a pregnant woman by the method of claim 12 and separating or isolating the identified stem cells for use in the diagnosis of prenatal genetic disorders.

18. A method for diagnosing chromosomal abnormality in a fetus which comprises: selecting at least one embryonic stem cell derived from the fetus using a germ cell specific marker; producing a display of the chromosomes of the embryonic stem cell and, analyzing the displayed chromosomes for abnormalities.

19. The method of claim 18 wherein the germ cell specific marker comprises a DAZL protein or a DAZL polynucleotide.

20. A method for identifying a selective embryonic stem cell marker which comprises: selecting a germ stem cell marker exhibiting specific expression in germ cells and absence of expression in differentiated somatic cells; and confirming the expression of the stem cell marker in embryonic stem cells.

21. A kit for enrichment and detection of fetal cells within a specimen, comprising: at least one reagent comprising an antibody to detect DAZL protein or a probe specific for DAZL RNA; and instructions for performing stem cell enrichment using the reagent, optionally including means for performing stem cell enrichment.

22. The kit of claim 21 further comprising reagents for genetic analysis of fetal and maternal cells.

23. The kit of claim 21 wherein the means for performing stem cell enrichment comprises at least one density gradient that concentrates fetal cells.

24. The kit of claim 21 wherein the reagent is a labeled with a detectable tracer.
